(19) United States
(12) Patent Application Publication
(10) Pub. No.: US 2026/0073336 A1
(43) Pub. Date: Mar. 12, 2026

(54) CLOUD-BASED QUALITY CONTROL DATA MANAGEMENT

(71) Applicant: Bio-Rad Laboratories, Inc., Hercules, CA (US)

(72) Inventors: Jasmina Jaswantrai DESAI, Irvine, CA (US); Mustafa Musa IBRAHIM, Placentia, CA (US)

(21) Appl. No.: 18/872,026

(22) PCT Filed: Jun. 1, 2023
(86) PCT No.: PCT/US2023/067787

§ 371 Date: Dec. 5, 2024

Related U.S. Application Data
(60) Provisional application No. 63/349,805, filed on Jun. 7, 2022.

(51) Int. Cl. G06Q 10/0639; G06Q 10/20; G16H 40/40; G16H 40/67

(52) U.S. Cl. CPC G06Q 10/06395; G06Q 10/20; G16H 40/40; G16H 40/67

(57) ABSTRACT

One or more instruments generate test result data. The test result data include patient data and QC data. The test result data is provided to a QC data flow system via a local network, which filters the test result data (e.g., using a set of rules) to extract the QC data. The QC data is provided to a cloud-based QC data management platform via an external network. The cloud-based QC data management platform analyzes the QC data and provides a result back to the QC data flow system. The QC data flow system forwards the result to middleware or the instrument, which triggers a corrective action based on the result as appropriate.

WO

### CROSS REFERENCE TO RELATED APPLICATIONS

The present application claims the benefit of U.S. Provisional Ser. No. 63/349,805 filed on Jun. 7, 2022, which is incorporated by reference.

### BACKGROUND

### 1. Technical Field

The subject matter described relates generally to managing instruments and, in particular, to using a management device to mediate communications between an instrument and a cloud-based Quality Control (QC) data management system.

### 2. Background Information

Laboratory instruments (e.g., clinical diagnostic instruments) generate QC data indicating the current operational status of the instruments. A QC sample for which an expected result is known may be periodically analyzed by an instrument and the actual result monitored over time. Thus, trends or changes in the operational status of the instrument may be detected, predicted, or corrected for. For example, calibration drift may be identified and corrected for or used to select an appropriate time to trigger a recalibration procedure. As another example, changes in the QC data over time may be used to predict when one or more reagents will need replacing. It should be appreciated that a wide range of operations may be performed based on QC data to improve the reliability and functionality of instruments.

However, existing approaches to QC data management are time consuming and prone to error. In one class of existing solutions, a human operator manually transfers QC data from an instrument to an on-site QC data management system (e.g., by copying the QC data to a USB drive and then uploading it to the QC data management system). This approach is prone to human error and is vulnerable to the responsible person forgetting to transfer the QC data or editing the QC data before uploading it. In another class of existing solutions, QC data is transferred to an on-site QC data management system via a local network. These approaches are problematic because the QC data management software needs to be customized for each site, making it difficult to provide support, maintenance, and updates.

### SUMMARY

The above and other problems may be solved by using an on-site QC data flow system in conjunction with a cloud-based QC data management platform. In various embodiments, the QC data flow system receives QC data from one or more instruments either directly or via a Laboratory Information System (LIS) or other middleware. The QC data flow system filters the data according to one or more rules and provides the filtered data to the cloud-based QC data management platform. The cloud-based QC data management platform analyzes the received QC data and provides responses to the QC data flow system, which may forward the responses to the corresponding instruments. For example, a response may indicate that an instrument is in good working order and no actions are required or that maintenance or other corrective action is required. The use of a single QC data flow system may encourage adoption as the instruments themselves need not interact directly with the cloud. Thus, so long as the QC data flow system is trusted to be secure, the advantages of cloud processing can be realized with less of the security concerns that typically go along with such services.

This architecture may enable uniform software and/or hardware to be used on-site, with the relevant customization being controlled remotely by the cloud-based QC data management platform. The QC data may be uploaded in a format agnostic manner, enabling new data formats and instruments to be supported without making significant on-site changes. Specifically, users may use a browser or custom app that is the same for all sites to review and manage QC data. In some embodiments, the QC data flow system provides bi-directional communication. Thus, a user may provide a configuration update to the QC data management platform, which pushes the update to the QC data management platform to be implemented.

### BRIEF DESCRIPTION OF THE DRAWINGS

FIG. 1 is a block diagram of a networked computing environment suitable for providing cloud-based QC data management, according to one embodiment.

FIG. 2 is a block diagram of the QC data flow system of FIG. 1, according to one embodiment.

FIG. 3 is a block diagram of the QC data management platform of FIG. 1, according to one embodiment.

FIG. 4 is a flowchart of a method for providing cloud-based QC data management, according to one embodiment.

FIG. 5 is a block diagram of an example of a computer suitable for use in the networked computing environment of FIG. 1, according to one embodiment.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### Example Systems

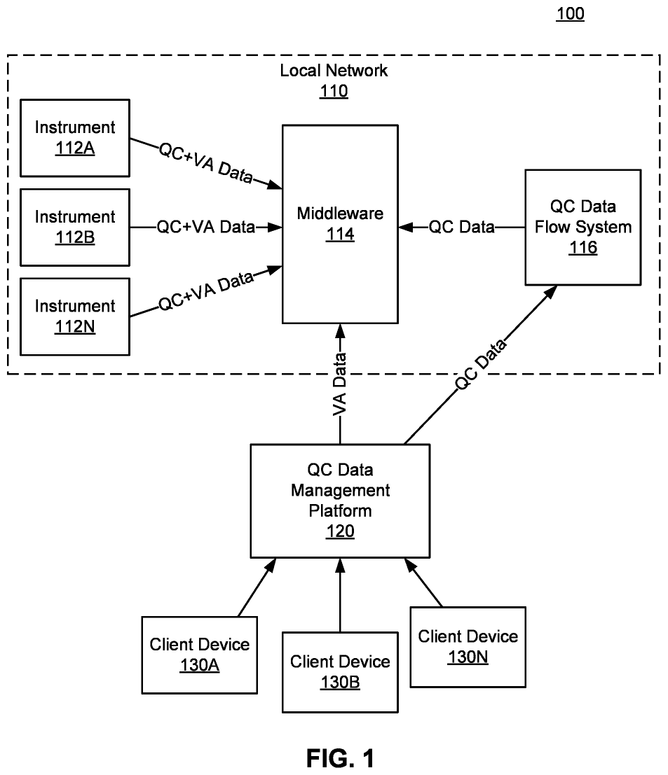

FIG. 1 illustrates one embodiment of a networked computing environment 100 suitable for providing cloud-based QC data management. In the embodiment shown, the networked computing environment 100 includes a local network 110, a QC data management platform 120, and one or more client devices 130. The local network 110 includes one or more instruments 112, middleware 114, and a QC data flow system 116. The local network 110 and client devices 130 communicate with the QC data management platform 120 via an external network (e.g., the internet). In other embodiments, the networked computing environment 100 includes different or additional elements. In addition, the functions may be distributed among the elements in a different manner than described.

The local network 110 is a computer network maintained by a laboratory or other entity to provide connectivity between the instruments 112, middleware 114, and QC data flow system 116. The physical infrastructure of the local network 110 will typically be in a single geographic location (e.g., a hospital or testing laboratory) but may also span multiple geographic locations (e.g., multiple laboratories connected by a VPN).

In the embodiments described below, the instruments 112 are clinical diagnostic instruments that analyze patient samples to generate test results or lab instruments that analyze samples to generate test results or other instruments that generate data. The test results typically include patient results obtained from analyzing patient samples and QC data. The QC data may include data generated from analyzing QC samples or running the instrument without a sample. Additionally or alternatively, the QC data may include data generated from or derived from test results. The QC data may include data the user deems relevant for QC purposes. The QC data may include additional information about the operation of the instrument 112. The instruments 112 may also generate Value Assignment (VA) data that may be used for instrument calibration. Although FIG. 1 shows three instruments (112A, 112B, and 112N), it should be appreciated that the local network 110 can include any number of instruments 112. It should also be appreciated that in other embodiments, other types of instruments may be managed using the same or similar technique as described below.

The instruments 112 provide test result data (including QC data) to the middleware 114. The middleware 114 provides software functionality within the local network 110 and manages operation of the instruments 112. For example, the middleware 114 may be part of a LIS that provides test results from patients for display to physicians and other operators. In the embodiment shown, the middleware 114 provides the test result data to the QC data flow system 116 and the VA data (if used) from the QC data management platform 120 to the relevant instruments 112 or other lab systems. Alternatively, one or more of the instruments 112 may provide test result data directly to the QC data flow system 116. In another alternative embodiment, VA data may be directly dispersed to one or more instruments 112 from the QC data flow system 116.

The QC data flow system 116 is an on-site resource that processes test result data and forwards it to the QC data management platform 120. The QC data flow system 116 may be a physical computing system or a virtual machine (e.g., running on a LIS) that acts as an edge node for the local network 110. In one embodiment, the QC data flow system 116 filters the test result data to extract the QC data and remove sensitive information, such as patient test results and other personal data. For example, the QC data flow system 116 may be configured to recognize the format of the test result data and extract the QC data. The QC data flow system 116 may be configured to recognize one or more standardized (or non-standardized) data formats. Furthermore, the QC data flow system 116 may be remotely updated to recognize and process new data formats without requiring a technician to visit the laboratory site.

Regardless of the precise filtering mechanism used, the QC data flow system 116 sends the QC data to the QC data management platform 120. The QC data flow system 116 receives a response and forwards the response to the middleware 114 or instrument 112. For example, the response may indicate that the instrument 112 is in good working order and no actions are required or that maintenance or other corrective action is required. In some embodiments, the middleware 114 or instrument 112 may automatically trigger preventative maintenance or another corrective action based on the received response such as evaluating the instrument calibration values.

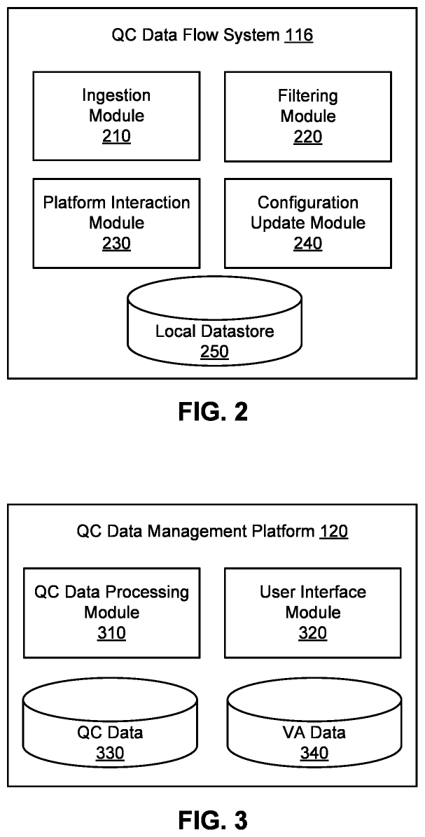

In some embodiments, the QC data flow system 116 enhances network security by disabling all of its ports except the ones used to communicate with the QC data management platform 120 and the middleware 114. Thus, external devices cannot use the QC data flow system 116 as a network entry point to the local network 110. Similarly, the QC data flow system 116 may encrypt some or all of the data sent via the external network to the QC data management platform 120. Various embodiments of the QC data flow system 116 are described in greater detail below, with reference to FIG. 2.

The QC data management platform 120 includes one or more cloud-based computer systems that process QC data and provide corresponding functionality to users. The QC data management platform 120 analyzes received QC data to determine the operable status of one or more instruments 112. In one embodiment, the QC data management platform 120 compares the received QC data for an instrument 112 to previously received QC data for that instrument to identify a pattern or trend. For example, if a measured value for a QC sample drops below or raises above a threshold, it may be indicative of a reagent supply needing to be refilled or a component of the instrument 112 needing replacement. Additionally or alternatively, the QC data management platform 120 may use a machine-learning model to identify the operable status of the instrument 112. The QC data management platform 120 may also provide a user interface (e.g., accessible via a client device 130) with which users can view results and make configuration updates. Various embodiments of the QC data management platform 120 are described in greater detail below, with reference to FIG. 3.

The client devices 130 are computing devices with which users can interact with the QC data management platform 120. FIG. 1 includes three client devices (130A, 130B, and 130N) but the networked computing environment 100 can include any number of client devices 130. In one embodiment, a client device 130 has a browser or dedicated app installed for interacting with the QC data management platform 120. By directing the browser to a web portal for the QC data management platform 120 (or opening the dedicated app) the user may be presented with a user interface for configuring the QC data flow system 116. For example, the user may submit a new data format for a new instrument 112 to enable the QC data flow system 116 to extract QC data from the test result data provided by the new instrument. The QC data management platform 120 may push the new configuration to the QC data flow system 116 for implementation without a technician having to visit the site at which the QC data flow system is located.

In some embodiments, the QC data management platform 120 may also provide the client device 130 with reports or notification on the operable status of instruments 112. For example, if maintenance is required for a particular instrument 112, a notification may be pushed to a client device 130 associated with a user responsible for the instrument. As another example, the client device 130 may display reports indicating trends on patterns in QC data for an instrument 112 for analysis by the user.

FIG. 2 illustrates one embodiment of the QC data flow system 116. In the embodiment shown, the QC data flow system 116 includes an ingestion module 210, a filtering module 220, a platform interaction module 230, a configuration update module 240, and a local datastore 250. In other embodiments, the QC data flow system 116 includes different or additional elements. In addition, the functions may be distributed among the elements in a different manner than described.

The ingestion module 210 receives test result data from one or more instruments. In one embodiment, the instruments 112 provide the test data to middleware 114 (e.g., provided by a LIS) and the middleware 114 pushes the test result data to the ingestion module 210. Alternatively, the ingestion module 210 may pull the test result data from the middleware 114. In either case, the test result data may be provided to the ingestion module 210: on receipt from an instrument 112, periodically (e.g., every five minutes, assuming there is new data available), when a certain amount of test result data has been generated, or on any other appropriate schedule.

The filtering module 220 filters the received test result data to extract QC data. In one embodiment, the filtering module 220 applies a set of one or more rules to the test result data to identify the QC data. For example, a first rule may identify a format of the test result data (e.g., based on an identifier of the instrument 112 that generated the test result data or by inspecting the test result data and comparing it to a set of templates of known data formats). A second rule may be selected based on the identified format and applied to extract the QC data. The second rule is specific to the data format and identifies which portion or portions of the test result data are QC data. It should be appreciated that a range of rule sets may be used to analyze the test result data and extract information of interest. For example, in some embodiments, the set of rules may also extract other data as well as QC data, such as extracting and transmitting instrument diagnostic data to help remotely diagnose an instrument or extracting molecular data for performing molecular diagnostics.

The platform interaction module 230 manages interactions between the QC data flow system 116 and the QC data management platform 120. In one embodiment, the platform interaction module 230 sends extracted QC data to the QC data management platform 120, which processes the QC data and sends a result back in response. The result may indicate the operable status of the instrument 112 that corresponds to the QC data. If the result indicates the instrument 112 is in good working order, the platform interaction module 230 may send confirmation to the instrument 112 (either via the middleware 114 or directly), causing the instrument to continue operating normally. In contrast, if the result indicates a problem, the platform interaction module 230 may send a signal to cause the instrument to pause operation and generate an alert (e.g., for display at a client device 130) that corrective action is needed, automatically trigger a corrective action (e.g., preventative maintenance), or both.

In some embodiments, the platform interaction module 230 sends a notification to the QC data flow system 116 in response to a triggering event. The triggering event may be a loss of connection to the QC data management platform 120 due to failure of the internet connection of the QC data flow system 116, the cloud, or any other disruption in service. Alternatively, the triggering event may be a user-input request to store QC data locally for set period of time. This may be advantageous in situations like a planned power outage, lab shutdowns, internet-impeding weather events, and other predicted disruptions

Regardless of the source of the triggering event, it causes the QC data flow system 116 to store QC data locally for up to maximum amount of time. The maximum amount of time may be user-defined or predetermined (e.g., 90 days). In one embodiment, the QC data flow system 116 stores the QC data until a connection to QC data management platform 120 is restored, at which time, the stored QC data is forwarded to the QC data management platform and then deleted from the QC data flow system 116. Alternatively, the QC data flow system 116 may store the QC data until the maximum amount of time expires or until a user provides input requesting otherwise, at which point, the QC data may be deleted without forwarding it to the QC data management platform 120.

The configuration update module 240 processes configuration updates received from the QC data management platform 120. In one embodiment, the configuration update module 240 receives updated or new rule sets and stores them for future use (e.g., in the local data store 250). In some embodiments, the configuration update module 240 may also distribute software or firmware to other devices within the local network 110. For example, a cloud system manager may maintain a list of registered edge devices securely and allow an authorized user to connect to a specific device remotely over a secure connection to update the configuration of the device.

The local datastore 250 includes one or more computer-readable media that store the data used by the QC data flow system 116. For example, the local datastore 250 may include one or more rule sets for processing QC data. Although the local datastore 250 is shown as a single entity that is part of the QC data flow system 116, in some embodiments, one or more external devices may be used for storage that are accessed via the local network 110.

FIG. 3 illustrates one embodiment of the QC data management platform 120. In the embodiment shown, the QC data management platform 120 includes a QC data processing module 310, a user interface module 320, QC data 330, and VA data 340. In other embodiments, the QC data management platform 120 includes different or additional elements. In addition, the functions may be distributed among the elements in a different manner than described.

The QC data processing module 310 receives and processes QC data from the QC data flow system 116. In one embodiment, the QC data processing module 310 receives new QC data from an instrument 112, identifies the instrument (e.g., based on an instrument ID in the new QC data), and retrieves historical QC data for the instrument (e.g., from the QC data 330). The QC data processing module 310 analyzes the new and historical QC data to determine the operable status of the instrument 112. For example, if the new QC data includes one or more values that differ by more than a threshold amount of percentage from a historical average or expected value, an error condition may be triggered. In some embodiments, the QC data processing module 310 may be configured to identify multiple different error conditions, each indicated by a corresponding pattern or variation from historical or expected values. Conversely, if the QC data is within one or more expected ranges, the QC data processing module 310 determines that the instrument 112 is in good working order. Regardless of exactly how the QC data is analyzed, the QC data processing module 310 sends an indication of the result of the analysis to the QC data flow system 116, which implements any required actions such as pausing operation of the instrument 112 or triggering preventative maintenance, etc.

The user interface module 320 provides a user interface that users may access via a client device 130. In one embodiment, the user interface enables a user to generate reports. A report may display some or all of the QC data 330 for one or more instruments 112 in an easily digestible format, e.g., data values over time may be plotted on charts to illustrate trends to enable the user to predict future needs.

In some embodiments, the user interface enables the user to provide new configurations for the QC data flow system 116 or instruments 112. For example, the user may provide a definition of a new data format to enable the QC data flow system 116 to extract QC data from the test result data generated by a new instrument 112. The user interface may also enable the user to remotely log in to the QC data flow system 116 or an instrument to provide remote support or maintenance.

The QC data 330 and VA data 340 are stored in one or more computer-readable media. The VA data 340 includes value assignments that can be used by the software controlling instruments 112 for calibration (e.g., to set ranges). Although the QC data 330 and VA data 340 are shown as distinct components, in some embodiments, they are stored together in a single data store. Furthermore, some or all of the QC data 330 and VA data 340 may be stored remotely and accessed via a network (e.g., in a distributed database).

### Example Methods

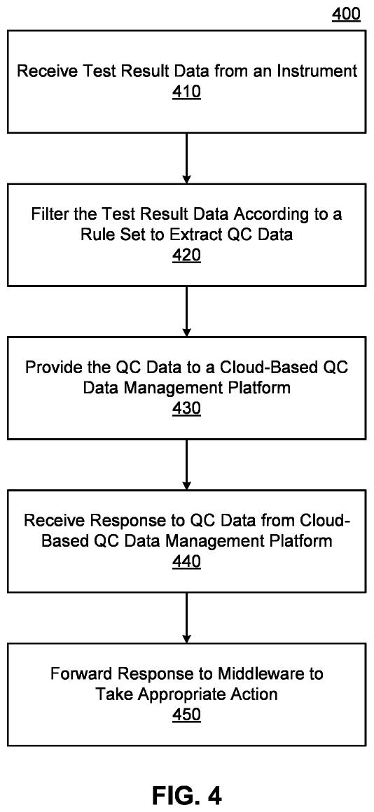

FIG. 4 illustrates a method 400 for providing cloud-based QC data management, according to one embodiment. The steps of FIG. 4 are illustrated from the perspective of the QC data flow system 116 performing the method 400. However, some or all of the steps may be performed by other entities or components. In addition, some embodiments may perform the steps in parallel, perform the steps in different orders, or perform different steps.

In the embodiment shown in FIG. 4, the method 400 begins with the QC data flow system 116 receiving 410 test result data from an instrument 112. The test result data may include results generated by analyzing patient samples and QC data. The QC data flow system 116 filters 420 the test result data using a set of one or more rules to extract the QC data. The QC data is provided 430 to the cloud-based QC data management platform 120 (e.g., via the internet). Because the test result data was filtered 420, any personal and health data of patients remains within the laboratory network 110.

The QC data flow system 116 receives 440 a response to the provided QC data from the QC data management platform 120. The response indicates the results of analysis on the QC data performed by the QC data management platform 120. For example, the response may indicate whether the instrument 112 is in good working order and can continue analyzing patient samples or whether maintenance or other corrective action should be taken. The QC data flow system 116 forwards 450 the response to the middleware 114 (e.g., a LIS) that takes appropriate action. If the response indicates that the instrument 112 is in good working order, appropriate action may be simply allowing the instrument to continue analyzing samples. Conversely, if the response indicates a problem, the appropriate action may include one or more of: causing the instrument 112 to stop analyzing samples, notifying a responsible user that corrective action is needed, or triggering a preventative maintenance operation. It should be appreciated that other types of corrective action are possible and may be recommended or automatically triggered by the middleware 114.

### Computing System Architecture

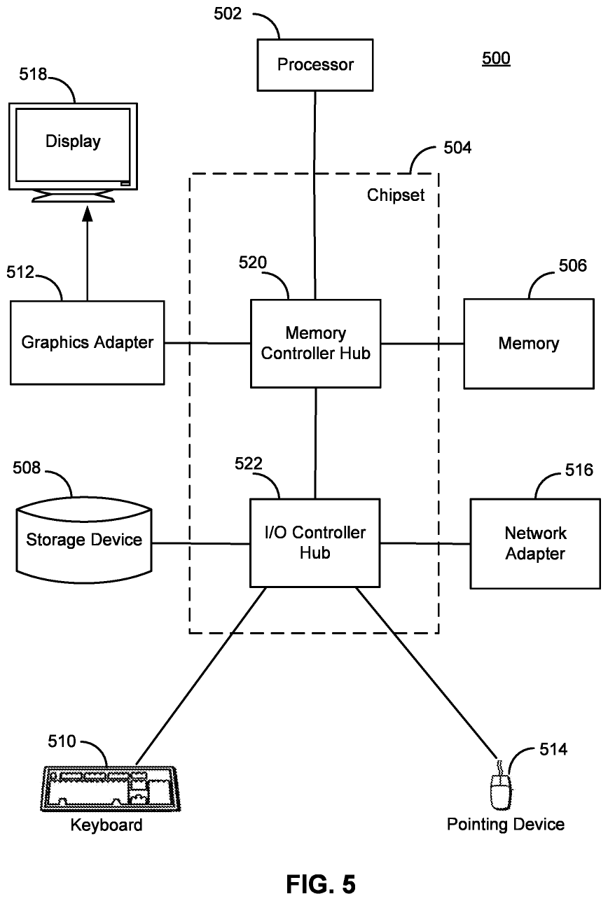

FIG. 5 is a block diagram of an example computer 500 suitable for use in the networked computing environment 100 of FIG. 1, according to one embodiment. The example computer 500 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, keyboard 510, pointing device 514, and network adapter 516 are coupled to the I/O controller hub 522. Other embodiments of the computer 500 have different architectures.

In the embodiment shown in FIG. 5, the storage device 508 is a non-transitory computer-readable storage medium such as a hard drive, compact disk read-only memory (CD-ROM), DVD, or a solid-state memory device. The memory 506 holds instructions and data used by the processor 502. The pointing device 514 is a mouse, track ball, touch-screen, or other type of pointing device, and may be used in combination with the keyboard 510 (which may be an on-screen keyboard) to input data into the computer system 500. The graphics adapter 512 displays images and other information on the display 518. The network adapter 516 couples the computer system 500 to one or more computer networks, such as the local network 110 or the external network.

The types of computers used by the entities of FIGS. 1 through 3 can vary depending upon the embodiment and the processing power required by the entity. For example, a LIS might include multiple blade servers working together to provide the functionality described. Furthermore, the computers can lack some of the components described above, such as keyboards 510, graphics adapters 512, and displays 518.

### Additional Considerations

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs for a system and a process fr providing cloud-based QC data management. Thus, while particular embodiments and applications have been illustrated and described, it is to be understood that the described subject matter is not limited to the precise construction and components disclosed. The scope of protection should be limited only by the following claims.

What is claimed is:

1. A computer-implemented method of providing cloud-based Quality Control (QC) data management, the method comprising:
receiving, via a local network, test result data for an instrument, the test result data including patient data and QC data;
filtering the test result data to extract the QC data;
providing, via an external network, the QC data to a cloud-based QC data management platform;
receiving, from the cloud-based QC data management platform, a response to the QC data, the response indicating an operable status of the instrument; and
providing the response, via the local network, to middleware that manages the instrument.

2. The computer-implemented method of claim 1, wherein a corrective action is triggered responsive to the response.

3. The computer-implemented method of claim 2, wherein the corrective action is automatically triggering preventative maintenance.

4. The computer-implemented method of claim 1, wherein the filtering is performed using a set of one or more rules.

5. The computer-implemented method of claim 4, further comprising:
receiving an updated rule set from the cloud-based QC data management platform;
receiving additional test result data for the instrument; and
extracting additional QC data from the additional test result data using the updated rule set.

6. The computer-implemented method of claim 1, wherein the instrument is a clinical diagnostic instrument.

7. The computer-implemented method of claim 1, further comprising:
identifying a triggering event indicating that connection to the cloud-based QC data management platform is unavailable;
responsive to the triggering event, storing the QC data on the local network for up to a maximum amount of time; and
responsive to receiving an indication that the connection to the cloud-based QC data management platform is available again, forwarding the stored QC data to the cloud-based QC data management platform.

8. The computer-implemented method of claim 7, further comprising, responsive to forwarding the QC data, deleting the QC data from the local network.

9. The computer-implemented method of claim 7, wherein the triggering event is user-input indicating planned downtime for the connection cloud-based QC data management platform.

10. A networked computing system for providing management of QC data, the networked computing system comprising:
one or more instruments that generate test result data, the test result data including patient data and QC data;
a Laboratory Information System (LIS) coupled to the one or more instruments via a local network;
a QC data flow system coupled to the LIS via the local network, the QC data flow system including one or more computing devices configured to receive the test result data and extract the QC data from the test result data using a set of one or more rules; and
a QC data management platform coupled to the QC data flow system via an external network, the QC data management platform including one or more computing devices configured to process the QC data to generate a result and send the result, via the external network, to the QC data flow system,
wherein the QC data flow system forwards the result to the LIS, and the LIS implements a corrective action for the instrument based on the result.

11. The networked computing system of claim 10, wherein the QC data flow system is a computing device located within a geographic space that includes the instrument.

12. The networked computing system of claim 10, wherein the QC data flow system is a virtual machine running on the LIS.

13. The networked computing system of claim 10, wherein all ports of the QC data flow system except those used to receive the test result data and provide the QC data to the QC data management platform are disabled.

14. The networked computing system of claim 10, further comprising:
identifying a triggering event indicating that connection to the cloud-based QC data management platform is unavailable;
responsive to the triggering event, storing the QC data on the local network for up to a set maximum of time; and
responsive to receiving an indication that the connection to the cloud-based QC data management platform is available again, forwarding the stored QC data to the cloud-based QC data management platform.

15. The networked computing system of claim 14, further comprising, responsive to forwarding the QC data, deleting the QC data from the local network.

16. The networked computing system of claim 14,
wherein the triggering event is user-input indicating planned downtime for the connection cloud-based QC data management platform.

17. A non-transitory computer-readable medium configured to store code comprising instructions, wherein the instructions, when executed by one or more processors, cause the one or more processors to perform steps comprising:
receiving, via a local network, test result data for an instrument, the test result data including patient data and QC data;
filtering the test result data to extract the QC data;
providing, via an external network, the QC data to a cloud-based QC data management platform;
receiving, from the cloud-based QC data management platform, a response to the QC data, the response including an operable status of the instrument; and
providing the response, via the local network, to middleware that manages the instrument.

18. The non-transitory computer-readable medium of claim 17, wherein a corrective action is triggered responsive to the response.

19. The non-transitory computer-readable medium of claim 17, further comprising:
receiving an updated rule set from the cloud-based QC data management platform;
receiving additional test result data for the instrument; and
extracting additional QC data from the additional test result data using the updated rule set.

20. The non-transitory computer-readable medium of claim 17, further comprising:
Identifying a triggering event indicating that connection to the cloud-based QC data management platform is unavailable;
responsive to the triggering event, storing the QC data on the local network for up to a maximum amount of time; and
responsive to receiving an indication that the connection to the cloud-based QC data management platform is available again, forwarding the stored QC data to the cloud-based QC data management platform.
